(19) United States
(12) Patent Application Publication
(10) Pub. No.: US 2015/0094777 A1
(43) Pub. Date: Apr. 2, 2015

(54) BONE SCREW

(71) Applicant: Exsomed Holding Company LLC, Scottsdale, AZ (US)

(72) Inventors: Lloyd P. Champagne, Phoenix, AZ (US); Jozef Zoldos, Phoenix, AZ (US)

(21) Appl. No.: 14/503,157

(22) Filed: Sep. 30, 2014

Related U.S. Application Data
(60) Provisional application No. 61/885,661, filed on Oct. 2, 2013.

(51) Int. Cl. A61B 17/86

(52) U.S. Cl. CPC A61B 17/8625
USPC 606/310

(57) ABSTRACT

Bone screws suitable for use with treatment of bones such as phalanges, particularly distal or terminal phalanges, and methods of using the bone screws are disclosed. Exemplary bone screws include a shaft having a thread about at least a portion of the shaft and at least one blade attached to the shaft. The at least one blade can be configured to bury into a bone to capture bone fragments and/or to securely attach to a bone.

### FIELD OF THE DISCLOSURE

The present disclosure relates to bone screws and to methods of using same.

### BACKGROUND OF THE DISCLOSURE

Bone screws have a variety of applications. For example, bone screws are often used to fix bones or portions thereof in place and/or to maintain proper alignment of bones. In the case of treatment of bone fractures in hands in particular, bone screws can be used for, for example, holding bone fragments in place as the bone is repaired.

A typical bone screw includes a shaft having a first end, which is initially inserted into a bone, and a second end, having a head. The head allows application of compression to a bone and/or fragments to be treated. In addition, the head of the screw prevents the bone screw from penetrating beyond a certain depth into the bone.

Although such bone screws work relatively well for some applications, including treatment of some fractures of phalanx bones, such bone screws do not work well for the treatment of fractures of the distal phalanx. As a result, standard screws are generally not used to repair fractures of distal phalanx. If a screw without a head is used to treat the fractured bone, desired compression on the bone cannot be achieved and the screw may wander (i.e., the screw may bury further into the bone or retreat from the bone) after initial placement of the screw. Accordingly, improved bone screws, including bone screws suitable for use with the distal phalanx, are desired.

### SUMMARY OF THE DISCLOSURE

Various embodiments of the disclosure relate to bone screws suitable for use in treatment of bone fractures. The bones screws described herein are particularly well suited for the treatment of fractures of phalanges, and particularly of fractures of the distal phalanx. While the ways in which the various embodiments of the disclosure address the drawbacks of the prior art are discussed in greater detail below, in general, exemplary bone screws include one or more blades that can bury into a bone and do not include a traditional screw head. The blades allow the bone screw to apply compression to fractured bone and mitigate migration of the bone screw after cannulation.

In accordance with exemplary embodiments of the disclosure, a bone screw includes a shaft having a first end, a second end, and a lumen extending through the shaft between the first end and the second end, a thread about a perimeter of the shaft, and at least one blade coupled to the shaft. In accordance with various aspects of these embodiments, the bone screw includes a plurality of blades—e.g., 2-12, 3-10, 4-8, or 6 blades. The thread can extend from a distance from a first end to a distance from the second end, such that the shaft includes a portion that may not include a thread. The distance from the first and/or second end may be, for example, from about 0 mm to about 2 mm, or about 0.25 to about 1 mm, or about 0.5 mm to about 1.5 mm. The outer diameter of the thread can range from, for example, 0.1 mm to about 1 mm, about 0.25 mm to about 0.75 mm, or about 0.5 mm greater than the outer diameter of the shaft. A diameter of the lumen can be configured to allow a wire, such as a k-wire, to be inserted therein and can have a diameter of about 0.05 mm to about 1.5 mm, or about 0.07 to about 1.2 mm, or about, or 0.1 mm to about 1 mm. The blades generally extend outward and have an outer radius that is larger than the outer radius or distance of the shaft and the thread. For example, the blade can have a diameter of about 0.025 to about 2 mm greater than the outer diameter of the thread, or about 0.5 to about 1.5 mm greater than the outer diameter of the thread, or about 0.75 to about 1.25 mm greater than the outer diameter of the thread. The blades may be straight or curved and have a cutting edge that may be tapered. The blade can include a leading or cutting edge, wherein a portion of the leading or cutting edge is closer to the first end (tip) compared to the second, distal end of the bone screw. Further the cutting and/or trailing edges can vary in distance relative to the first end to facilitate cutting of the blade(s) into a bone. In accordance with further aspects of these embodiments, the bone screw is configured to bury at least one blade about 0.1 to about 2 mm, 0.25 to about 1, or about 0.5 mm into a bone. The bone screw and components thereof may be made of any suitable material, such as metal (e.g., titanium or steel), ceramic, biodegradable polymer, other polymer material, drug eluting material, or any combination thereof. The bone screw may be integrally formed or one or more parts may be separately formed and assembled together.

In accordance with additional exemplary embodiments of the invention, a bone screw for treatment of the distal phalanx includes a shaft having a first end and a second end, a thread about a perimeter of the shaft, and at least one blade coupled to the shaft. The bone screw may further include a lumen spanning between the first end and the second end. In accordance with various aspects of these embodiments, the bone screw includes a plurality of blades—e.g., 2-12, 3-10, 4-8, or 6 blades. The thread can extend from a distance from the first end to a distance from the second end, such that the shaft includes a portion that does not include a thread. The distance may be, for example, from about 0 mm to about 2 mm, or about 0.25 to about 1 mm, or about 0.5 mm to about 1.5 mm, and may be the same or different from either end. The outer diameter of the thread can range from, for example, 0.1 mm to about 1 mm, about 0.25 mm to about 0.75 mm, or about 0.5 mm greater than the outer diameter of the shaft. A diameter of the lumen can be about 0.05 mm to about 1.5 mm or about 0.07 to about 1.2 mm, or about, or 0.1 mm to about 1 mm. The blades can have a diameter of about 0.025 to about 2 mm greater than the outer diameter of the thread, or about 0.5 to about 1.5 mm greater than the outer diameter of the thread, or about 0.75 to about 1.25 mm greater than the outer diameter of the thread. The blades may be straight or curved and have a cutting edge that may be tapered. The blades can include a leading or cutting edge, wherein a portion of the leading or cutting edge is closer to the first end (tip) than to the second, distal end of the bone screw. Further the cutting and/or trailing edges can vary in distance relative to the first end to facilitate cutting of the blade(s) into a bone. In accordance with further aspects of these embodiments, the bone screw is configured to bury at least one blade about 0.1 to about 2 mm, 0.25 to about 1, or about 0.5 mm into a bone. The bone screw and components thereof may be made of any suitable material, such as metal (e.g., titanium or steel), ceramic, biodegradable polymer, other polymer material, drug eluting material, or any combination thereof. The bone screw may be integrally formed or one or more parts may be separately formed and assembled together.

In accordance with yet other embodiments of the invention, a bone screw for treatment of a phalanx includes a shaft having a first end and a second end, a thread about a perimeter of the shaft, and a plurality of blades coupled to the shaft. The bone screw may further include a lumen spanning between the first end and the second end. In accordance with various aspects of these embodiments, the bone screw includes a plurality of blades—e.g., 2-12, 3-10, 4-8, or 6 blades. The thread can extend from a distance from the first end to a distance from the second end, such that the shaft can include a portion that does not includes a thread. The distance may be, for example, from about 0 mm to about 2 mm, or about 0.25 to about 1 mm, or about 0.5 mm to about 1.5 mm. The outer diameter of the thread can range from, for example, 0.1 mm to about 1 mm, about 0.25 mm to about 0.75 mm, or about 0.5 mm greater than the outer diameter of the shaft. A diameter of the lumen can be about 0.05 mm to about 1.5 mm or about 0.07 to about 1.2 mm, or about, or 0.1 mm to about 1 mm. The blades can have a diameter of about 0.025 to about 2 mm greater than the outer diameter of the thread, or about 0.5 to about 1.5 mm greater than the outer diameter of the thread, or about 0.75 to about 1.25 mm greater than the outer diameter of the thread. The blades may be straight or curved and have a cutting edge that may be tapered. The blade can include a leading or cutting edge, wherein a portion of the leading or cutting edge is closer to the first end (tip) than to the second, distal end of the bone screw. Further the cutting and/or trailing edges can vary in distance relative to the first end to facilitate cutting of the blade(s) into a bone. In accordance with further aspects of these embodiments, the bone screw is configured to bury at least one blade about 0.1 to about 2 mm, 0.25 to about 1, or about 0.5 mm into a bone. The bone screw and components thereof may be made of any suitable material, such as metal (e.g., titanium or steel), ceramic, biodegradable polymer, other polymer material, drug eluting material, or any combination thereof. The bone screw may be integrally formed or one or more parts may be separately formed and assembled together.

### BRIEF DESCRIPTION OF THE DRAWINGS

A more complete understanding of exemplary embodiments of the present disclosure may be derived by referring to the detailed description and claims when considered in connection with the following illustrative figures.

FIG. 1 illustrates a bone screw in accordance with exemplary embodiments of the disclosure.

FIG. 2 illustrates a bone screw inserted into a bone in accordance with further exemplary embodiments of the disclosure.

FIG. 3 illustrates a top view of a bone screw in accordance with yet additional exemplary embodiments of the disclosure.

FIG. 4 illustrates a top view of a bone screw in accordance with exemplary embodiments of the disclosure.

It will be appreciated that the figures are not necessarily drawn to scale. For example, the dimensions of some of the elements in the figures may be exaggerated relative to other elements to help to improve understanding of illustrated embodiments of the present invention.

### DETAILED DESCRIPTION OF EXEMPLARY EMBODIMENTS

The description of exemplary embodiments of the present invention provided below is merely exemplary and is intended for purposes of illustration only; the following description is not intended to limit the scope of the invention disclosed herein. Moreover, recitation of multiple embodiments having stated features is not intended to exclude other embodiments having additional features or other embodiments incorporating different combinations of the stated features.

The present disclosure describes bone screws that are suitable for use in treatment of fractured bones. The bones screws are conveniently described below in connection with treatment of a distal phalanx; however, exemplary bone screws described herein are not limited to such applications, unless otherwise noted herein.

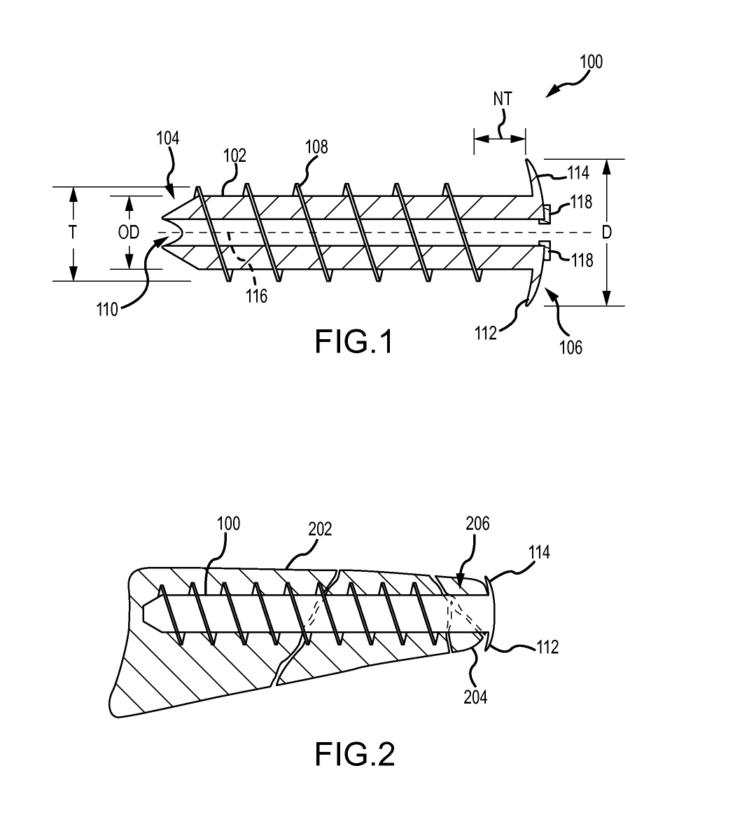

FIG. 1 illustrates a bone screw 100 in accordance with exemplary embodiments of the disclosure. Bone screw 100 includes a shaft 102, having a first end 104 and a second end 106; a thread 108 about a perimeter of shaft 102, a lumen 110 spanning between first end 104 and second end 106, and a at least one blade 112, 114. Bone screw 100 is configured to be inserted into a bone, such as a distal phalanx, such that the blades 112, 114 can be buried into the bone. Blades 112, 114 facilitate capture and retention of bone material/fragments, allow desired compression to be applied to the bone for treatment of the fracture, and burying blades 112, 114 prevents or mitigates the risk of the screw moving further into or out of the bone after placement of bone screw 100 in a desired location. In addition, because blades 112, 114 are buried, bone screw 100, once fully inserted, does not extend beyond or minimally extends beyond the surface of the bone.

Shaft 102 may be formed of any suitable material. For example, shaft 102 may be formed of metal, such as titanium or stainless steel, such as high-grade carbon steel, ceramic material, biodegradable polymer material, other polymer material, drug eluting material, and various combinations of such material. A length and width of shaft 102 may vary according to application and/or on a patient and/or bone thereof to be treated. By way of examples, a length of shaft 102 ranges from about 1 to 10 mm, 2-8 mm, or 5-6 mm, and an outer 102 (illustrated as OD in FIG. 1) can range from about 0.5 to about 5 mm, about 1 to about 3 mm, or about 2 mm. Shaft 102 includes a central axis 116.

Lumen 110 within shaft 102 can be configured to receive a guide wire, such as a k-wire, used for treatment of bones. Exemplary diameters for lumen 110 include about 0.05 to about 1.5 mm, or about 0.07 to about 1.2 mm, or about 0.9 mm. Lumen 110 also includes a central axis. In the illustrated example, bone screw 100 includes a central axis 116 that is common to both shaft 102 and lumen 110; however bone screws in accordance with this disclosure are not so limited.

Bone screw 100 includes at least one and optionally a plurality of blades 112, 114. By way of examples, bone screw 100 includes 1-12, 3-10, 4-8, or six or more blades (two blades 112, 114 are illustrated in FIG. 2). Blades 112, 114 may be formed of any suitable material, such as any of the materials noted herein in connection with bone screw 100. Blades 112, 114 may be flat or substantially flat, or have a curvature with a radius perpendicular an axis 116 , parallel axis 116, or a radius along a direction that is neither parallel nor perpendicular axis 116—e.g., with a curvature similar to a typical fan blade. In accordance with specific examples, blade 112 and/or 114 includes a cutting edge that can include a tapered (e.g., sharpened) edge to facilitate cutting through the bone. In accordance with further examples, a portion of a cutting edge (e.g., greater than 90%, 80%, 70%, 60%, 50%, 40%, 30%, 20%, or 10%) of the at least one blade is closer the first end than a trailing edge of the at least one blade.

An outer diameter of blade 112, 114 is generally greater than an outer diameter of thread 108. For example, the blade diameter (defined as a distance from an outer edge of one blade to a central axis of shaft 102 multiplied by 2) can be about 0.025 to about 2 mm greater than the outer diameter of the thread, about 0.5 to about 1.5 mm greater than the outer diameter of the thread, 0.75 to about 1.25 mm greater than the outer diameter of the thread, or about 1 mm greater than the outer diameter of the thread. By way of specific examples, a distance tip-to-tip of opposing blades 112, 114, illustrated as D in FIG. 1, can be about 2-5 mm, about 3-4 mm, or about 3.5 mm.

Thread 108 can be integrally formed with shaft 102 and can extend from first end 104 or from a distance from first end 104 to second end 106 or a distance from second end 106. The distance from either end can be, for example, from 0, about 0.1 mm to about 2 mm, or from about 0.25 mm to about 1 mm. An area NT on FIG. 1 illustrates an area near second end 106 with no thread on shaft 102.

Exemplary outer diameters of thread 108 include about 0.1 mm to about 1 mm greater than an outer diameter of the shaft, about 0.25 mm to about 0.75 mm greater than the outer diameter of the shaft, or about 0.5 mm greater than the outer diameter of the shaft. A total diameter of thread 108, illustrated by T in FIG. 1, can be about 1-6 nm, 2-3.5 mm, or about 2.5 mm.

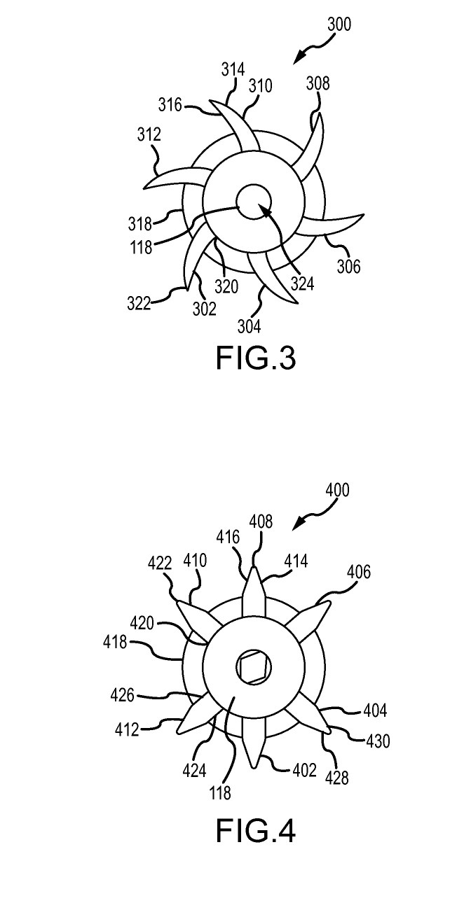

Bone screw 100 can also include a coupling element 118 to couple blades 112, 114 to shaft 102. When bone screw 100 includes coupling element 118, coupling element 118 can be formed of any suitable material, such as the materials described herein in connection with bone screw 100 and components thereof. Alternatively, blades 112, 114 and shaft and/or other bone screw 100 components and various combinations of components can be formed as an integral screw or component. Coupling element 118, better illustrated in FIGS. 3 and 4 can be configured to receive a tool, such as a wrench (e.g., allen), within a recess 324, to move bone screw 100 to a desired location within a bone.

Turning now to FIG. 2 a bone screw, such as bone screw 100, partially inserted into a fractured distal phalanx 202, is illustrated. In the illustrated example, blades 112, 114 are not bored into a location underneath a surface 204 of distal phalanx 202. In this case, bone screw 100 may be further inserted, such that blades 112, 114 are under surface 204 or a portion of blades 112, 114 may stay above surface 204. When blades 112, 114 are inserted to an area 206 beneath surface 204, blades 112, 114 can be buried at a distance of about 0.1 to about 2 mm into a bone, 0.25 to about 1 mm into the bone, or about 0.5 mm or more from surface 204 into the bone.

Turning now to FIGS. 3 and 4, exemplary top views of bone screws 300 and 400 are illustrated. In the illustrated embodiments, bone screw 300 includes six blades 302-312 attached to a shaft having a thread 318 thereon. Each blade 302-312 includes a cutting edge 314 and a trailing edge 316. However, other bone screws in accordance with the present disclosure may include blades where not all of the blades have a cutting and/or tailing edge. Each of cutting edge 314 and trailing edge 316 include a radius of curvature, for example ranging from about 0.25 to about 5 mm. Further, cutting edge 314 and optionally trailing edge 316 may be tapered or sharpened to facilitate burying of screw 300 into a bone. Blades 302-312 can also be bent, as noted above, such that a portion of the leading or cutting edge (e.g., greater than 90%, 80%, 70%, 60%, 50%, 40%, 30%, 20%, or 10%) is closer to the tip (e.g., end 104 illustrated in FIG. 1) relative to trailing edge 316. Blades 302-312 include a base 320 and a tip 322, wherein a width of the blade tapers (e.g., continuously or substantially continuously) from the base to the tip.

Bone screw 400 is also illustrated with six blades 402-412 attached to a shaft having a thread 418 thereon. Similar to blades 302-312, blades 402-412 can include a leading or cutting edge 414 and a trailing edge 416. Cutting edge 414 and optionally trailing edge 416 can be tapered or sharpened to facilitate burying screw 400 into a bone. In the example illustrated in FIG. 4, blades 402-412 are flat. Blades 402-412 include a base 420 and a tip 422, wherein a width of the blade tapers from the base to the tip. In the illustrated example, blades 402 include straight sections 424, 426 and tapering (e.g., continuously or substantially continuously) sections 428, 430.

In accordance with other embodiments, a bone screw (e.g., bone screw 100, 300, or 400) for treatment of the distal phalanx includes a shaft having a first end and a second end, a thread about a perimeter of the shaft, and at least one blade coupled to the shaft. The bone screw may further include a lumen spanning between the first end and the second end.

In accordance with yet further embodiments, a bone screw (e.g., bone screw 100, 300, or 400) for treatment of a phalanx includes a shaft having a first end and a second end, a thread about a perimeter of the shaft, and a plurality of blades coupled to the shaft. The bone screw may further include a lumen spanning between the first end and the second end.

Some specific examples of the invention are described below:

1. A bone screw comprising:
a shaft having a first end, a second end, and a lumen extending through the shaft between the first end and the second end;
a thread about a perimeter of the shaft; and
at least one blade coupled to the shaft.

2. The bone screw of example 1, wherein the bone screw comprises a plurality of blades.

3. The bone screw of any of examples 1-2, wherein the bone screw comprises 1-12 blades.

4. The bone screw of any of examples 1-3, wherein the bone screw comprises 3-10 blades. 5. The bone screw of any of examples 1-4, wherein the bone screw comprises 4-8 blades. 6. The bone screw of any of examples 1-5, wherein the bone screw comprises 6 blades.

7. The bone screw of any of examples 1-6, wherein the thread extends from a first distance from the first end to a second distance from the second end.

8. The bone screw of example 7, wherein the second distance ranges from about 0.1 mm to about 2 mm.

9. The bone screw of example 7, wherein the second distance ranges from about 0.25 mm to about 1 mm.

10. The bone screw of any of examples 1-9, wherein the lumen has a diameter of about 0.05 to about 1.5 mm.

11. The bone screw of any of examples 1-10, wherein the lumen has a diameter of about 0.07 to about 1.2 mm.

12. The bone screw of any of examples 1-11, wherein an outer diameter of the thread is about 0.1 mm to about 1 mm greater than an outer diameter of the shaft.

13. The bone screw of any of examples 1-12, wherein the outer diameter of the thread is about 0.25 mm to about 0.75 mm greater than the outer diameter of the shaft.

14. The bone screw of any of examples 1-13, wherein the outer diameter of the thread is about 0.5 mm greater than the outer diameter of the shaft.

15. The bone screw of any of examples 1-14, wherein an outer diameter of the shaft is about 0.5 to about 5 mm.

16. The bone screw of any of examples 1-15, wherein an outer diameter of the shaft is about 1 to about 3 mm.

17. The bone screw of any of examples 1-16, wherein an outer diameter of the shaft is about 2 mm.

18. The bone screw of any of examples 1-17, wherein a diameter of the at least one blade is about 0.025 to about 2 mm greater than the outer diameter of the thread.

19. The bone screw of any of examples 1-18, wherein a diameter of the at least one blade is about 0.5 to about 1.5 mm greater than the outer diameter of the thread.

20. The bone screw of any of examples 1-19, wherein a diameter of the at least one blade is about 0.75 to about 1.25 mm greater than the outer diameter of the thread.

21. The bone screw of any of examples 1-20, wherein a diameter of the at least one blade is about 1 mm greater than the outer diameter of the thread.

22. The bone screw of any of examples 1-21, wherein the at least one blade comprises a cutting edge.

23. The bone screw of example 22, wherein the cutting edge is tapered.

24. The bone screw of any of examples 1-23, wherein the at least one blade has a radius of curvature perpendicular an axis of the lumen.

25. The bone screw of any of examples 1-24, wherein at least a portion of a cutting edge of the at least one blade is closer the first end than a trailing edge of the at least one blade.

26. The bone screw of any of examples 1-24, wherein more than half of a cutting edge of the at least one blade is closer the first end than at least half of a trailing edge of the at least one blade.

27. The bone screw of any of examples 1-24, wherein the blade is substantially flat.

28. The bone screw of any of examples 1-27, further comprising a coupling element coupled to the at least one blade and the shaft.

29. The bone screw of any of examples 1-27, wherein the shaft, the thread, and the at least one blade are integrally formed.

30. A bone screw for treatment of the distal phalanx, the bone screw comprising:
a shaft having a first end and a second end;
a thread about a perimeter of the shaft; and
at least one blade coupled to the shaft.

31. The bone screw of example 30, further comprising a lumen spanning between the first end and the second end.

32. The bone screw of any of examples 30-31, wherein the bone screw comprises a plurality of blades.

33. The bone screw of any of examples 30-32, wherein the bone screw comprises 1-12 blades.

34. The bone screw of any of examples 30-33, wherein the bone screw comprises 3-10 blades.

35. The bone screw of any of examples 30-34, wherein the bone screw comprises 4-8 blades.

36. The bone screw of any of examples 30-35, wherein the bone screw comprises 6 blades.

37. The bone screw of any of examples 30-36, wherein the thread extends from a first end to a distance from the second end.

38. The bone screw of example 37, wherein the distance ranges from about 0.1 mm to about 2 mm.

39. The bone screw of example 37, wherein the distance ranges from about 0.25 mm to about 1 mm.

40. The bone screw of any of examples 31-39, wherein the lumen has a diameter of about 0.05 to about 1.5 mm.

41. The bone screw of any of examples 31-40, wherein the lumen has a diameter of about 0.07 to about 1.2 mm.

42. The bone screw of any of examples 30-41, wherein an outer diameter of the thread is about 0.1 mm to about 1 mm greater than an outer diameter of the shaft.

43. The bone screw of any of examples 30-42, wherein an outer diameter of the thread is about 0.25 mm to about 0.75 mm greater than an outer diameter of the shaft.

44. The bone screw of any of examples 30-43, wherein an outer diameter of the thread is about 0.5 mm greater than an outer diameter of the shaft.

45. The bone screw of any of examples 30-44, wherein an outer diameter of the shaft is about 0.5 to about 5 mm.

46. The bone screw of any of examples 30-35, wherein an outer diameter of the shaft is about 1 to about 3 mm.

47. The bone screw of any of examples 30-36, wherein an outer diameter of the shaft is about 2 mm.

48. The bone screw of any of examples 30-37, wherein an outer diameter of the at least one blade is about 0.025 to about 2 mm greater than the outer diameter of the thread.

49. The bone screw of any of examples 30-38, wherein an outer diameter of the at least one blade is about 0.5 to about 1.5 mm greater than the outer diameter of the thread.

50. The bone screw of any of examples 30-39, wherein an outer diameter of the at least one blade is about 0.75 to about 1.25 mm greater than the outer diameter of the thread.

51. The bone screw of any of examples 30-40, wherein an outer diameter of the at least one blade is about 1 mm greater than the outer diameter of the thread.

52. The bone screw of any of examples 30-41, wherein the at least one blade comprises a cutting edge.

53. The bone screw of example 52, wherein the cutting edge is tapered.

54. The bone screw of any of examples 31-53, wherein the at least one blade has a radius of curvature perpendicular an axis of the lumen.

55. The bone screw of any of examples 30-54, wherein at least a portion of a cutting edge of the at least one blade is closer the first end than a trailing edge of the at least one blade.

56. The bone screw of any of examples 30-54, wherein more than half of a cutting edge of the at least one blade is closer the first end than at least half of a trailing edge of the at least one blade.

57. The bone screw of any of examples 30-54, wherein the blade is substantially flat.

58. The bone screw of any of examples 30-57, further comprising a coupling element coupled to the at least one blade and the shaft.

59. The bone screw of any of examples 30-54, wherein the shaft, the thread, and the at least one blade are integrally formed.

60. A bone screw for treatment of a phalanx, the bone screw comprising:
a shaft having a first end and a second end;
a thread about a perimeter of the shaft; and
a plurality of blades coupled to the shaft.

61. The bone screw of example 60, further comprising a lumen spanning between the first end and the second end.

62. The bone screw of example 60, wherein the bone screw is configured to bury the at least one blade about 0.1 to about 2 mm into a bone.

63. The bone screw of any of examples 60-61, wherein the bone screw is configured to bury the at least one blade about 0.25 to about 1 mm into a bone.

64. The bone screw of any of examples 60-62, wherein the bone screw is configured to bury the at least one blade about 0.5 mm or more into a bone.

65. The bone screw of any of examples 60-64, wherein the bone screw comprises 1-12 blades.

66. The bone screw of any of examples 60-65, wherein the bone screw comprises 3-10 blades.

67. The bone screw of any of examples 60-66, wherein the bone screw comprises 4-8 blades.

68. The bone screw of any of examples 60-67, wherein the bone screw comprises 6 blades.

69. The bone screw of any of examples 60-68, wherein the thread extends from a first end to a distance from the second end.

70. The bone screw of example 69, wherein the distance ranges from about 0.1 mm to about 2 mm.

71. The bone screw of any of examples 69-70, wherein the distance ranges from about 0.25 mm to about 1 mm.

72. The bone screw of any of examples 61-71, wherein the lumen has a diameter of about 0.05 to about 1.5 mm.

73. The bone screw of any of examples 61-72, wherein the lumen has a diameter of about 0.07 to about 1.2 mm.

74. The bone screw of any of examples 60-73, wherein an outer diameter of the thread is about 0.1 mm to about 1 mm greater than an outer diameter of the shaft.

75. The bone screw of any of examples 60-74, wherein an outer diameter of the thread is about 0.25 mm to about 0.75 mm greater than the outer diameter of the shaft.

76. The bone screw of any of examples 60-75, wherein the outer diameter of the thread is about 0.5 mm greater than the outer diameter of the shaft.

77. The bone screw of any of examples 60-76, wherein an outer diameter of the shaft is about 0.5 to about 5 mm.

78. The bone screw of any of examples 60-77, wherein the outer diameter of the shaft is about 1 to about 3 mm.

79. The bone screw of any of examples 60-78, wherein the outer diameter of the shaft is about 1.5 to 2 mm.

80. The bone screw of any of examples 60-79, wherein an outer diameter of at least one blade is about 0.025 to about 2 mm greater than the outer diameter of the thread.

81. The bone screw of any of examples 60-80, wherein an outer diameter of the at least one blade is about 0.5 to about 1.5 mm greater than the outer diameter of the thread.

82. The bone screw of any of examples 60-81, wherein an outer diameter of at least one blade is about 0.75 to about 1.25 mm greater than the outer diameter of the thread.

83. The bone screw of any of examples 60-82, wherein an outer diameter of at least one blade is about 1 mm greater than the outer diameter of the thread.

84. The bone screw of any of examples 60-83, wherein an outer diameter of at least one blade is about 2 to 5 mm.

85. The bone screw of any of examples 60-84, wherein an outer diameter of at least one blade is about 2.5 to 4 mm.

86. The bone screw of any of examples 60-85, wherein an outer diameter of at least one blade is about 3 to 3.5 mm.

87. The bone screw of any of examples 60-86, wherein the at least one blade comprises a cutting edge.

88. The bone screw of example 87, wherein the cutting edge is tapered.

89. The bone screw of any of examples 60-88, wherein at least one blade has a radius of curvature perpendicular an axis of the lumen.

90. The bone screw of any of examples 60-89, wherein at least a portion of a cutting edge of the at least one blade is closer the first end than a trailing edge of the at least one blade.

91. The bone screw of any of examples 60-90, wherein more than half of a cutting edge of the at least one blade is closer the first end than at least half of a trailing edge of the at least one blade.

92. The bone screw of any of examples 60-89, wherein the blade is substantially flat.

93. The bone screw of any of examples 60-92, further comprising a coupling element coupled to the at least one blade and the shaft.

94. The bone screw of any of examples 60-93, wherein the shaft, the thread, and the at least one blade are integrally formed.

95. The bone screw of any of examples 1-94, wherein the shaft comprises metal.

96. The bone screw of any of examples 1-94, wherein the shaft comprises titanium.

97. The bone screw of any of examples 1-94, wherein the shaft comprises a ceramic material.

98. The bone screw of any of examples 1-94, wherein the shaft comprises a metal and a polymer material.

99. The bone screw of any of examples 1-94, wherein the shaft comprises a biodegradable material.

100. The bone screw of any of examples 1-28, 30-58, or 60-99, wherein the shaft and blades are separately formed.

101. The bone screw of any of examples 1-23, 25-53, 55-88, and 90-100, wherein the at least one blade has a radius of curvature not perpendicular an axis of the shaft.

102. The bone screw of any of examples 1-100, wherein the at least one blade has a leading edge and a trailing edge, wherein the leading edge is tapered and the trailing edge is tapered.

The present invention has been described above with reference to a number of exemplary embodiments and examples. It should be appreciated that the particular embodiments shown and described herein are illustrative of the preferred embodiments of the invention and its best mode, and are not intended to limit the scope of the invention as set forth in the claims. It will be recognized that changes and modifications may be made to the embodiments described herein without departing from the scope of the present invention. These and other changes or modifications are intended to be included within the scope of the present invention, as expressed in the following claims and the legal equivalents thereof.

What is claimed is:

1. A bone screw comprising:
a shaft having a first end, a second end, and a lumen extending through the shaft between the first end and the second end;
a thread about a perimeter of the shaft; and
at least one blade coupled to the shaft.

2. The bone screw of claim 1, wherein the bone screw comprises a plurality of blades.

3. The bone screw of claim 1, wherein the bone screw comprises 1-12 blades.

4. The bone screw of claim 1, wherein the bone screw comprises 3-10 blades.

5. The bone screw of claim 1, wherein the bone screw comprises 4-8 blades.

6. The bone screw of claim 1, wherein the bone screw comprises 6 blades.

7. The bone screw of claim 1, wherein the thread extends from a first distance from the first end to a second distance from the second end.

8. The bone screw of claim 7, wherein the second distance ranges from about 0.1 mm to about 2 mm.

9. The bone screw of claim 7, wherein the second distance ranges from about 0.25 mm to about 1 mm.

10. The bone screw of claim 1, wherein the lumen has a diameter of about 0.05 to about 1.5 mm.

11. The bone screw of claim 1, wherein the lumen has a diameter of about 0.07 to about 1.2 mm.

12. The bone screw of claim 1, wherein an outer diameter of the thread is about 0.1 mm to about 1 mm greater than an outer diameter of the shaft.

13. The bone screw of claim 1, wherein the outer diameter of the thread is about 0.25 mm to about 0.75 mm greater than the outer diameter of the shaft.

14. The bone screw of claim 1, wherein the outer diameter of the thread is about 0.5 mm greater than the outer diameter of the shaft.

15. The bone screw of claim 1, wherein an outer diameter of the shaft is about 0.5 to about 5 mm.

16. The bone screw of claim 1, wherein an outer diameter of the shaft is about 1 to about 3 mm.

17. The bone screw of claim 1, wherein an outer diameter of the shaft is about 2 mm.

18. The bone screw of claim 1, wherein a diameter of the at least one blade is about 0.025 to about 2 mm greater than the outer diameter of the thread.

19. The bone screw of claim 1, wherein a diameter of the at least one blade is about 0.5 to about 1.5 mm greater than the outer diameter of the thread.

20. The bone screw of claim 1, wherein a diameter of the at least one blade is about 0.75 to about 1.25 mm greater than the outer diameter of the thread.
